(19) United States
(12) Patent Application Publication
(10) Pub. No.: US 2024/0033108 A1
(43) Pub. Date: Feb. 1, 2024

(54) METHOD OF CONTROLLING A JOINT OF AN ORTHOPAEDIC TECHNOLOGY DEVICE AND JOINT OF THIS KIND

(71) Applicant: Ottobock SE & Co. KGaA, Duderstadt (DE)

(72) Inventors: Eric Claussnitzer, Duderstadt (DE); Mark Schönemeier, Duderstadt (DE)

(21) Appl. No.: 18/258,399

(22) PCT Filed: Dec. 17, 2021
(86) PCT No.: PCT/EP2021/086619

§ 371 Date: Jun. 20, 2023

(30) Foreign Application Priority Data
Dec. 22, 2020 (DE) 10 2020 134 703.0

(51) Int. Cl. A61F 2/70; A61F 2/66; A61F 2/64

(52) U.S. Cl. CPC A61F 2/70; A61F 2/6607; A61F 2/64; A61F 2002/607

(57) ABSTRACT

The invention relates to a method for controlling a joint (2, 28) of an orthopedic device that comprises a first part (8), a second part (4), which is arranged on the first part (8) such that it can be pivoted about a pivot axis (12), an active actuator (42), a self-locking transmission (16, 50) and an electric control unit for controlling the actuator (42), the electric control unit controlling the actuator (42) during the method in such a way that the second part (4) moves according to forces acting on it externally.

The invention relates to a method for controlling a joint of an orthopedic device that comprises a first part, a second part, which is arranged on the first part such that it can be pivoted about a pivot axis, an active actuator, a self-locking transmission and an electric control unit for controlling the actuator. The invention also relates to an artificial joint of this kind.

An orthopedic device is, in particular, an orthosis or a prosthesis. Support devices that, for example, support work carried out above the head or prevent tiredness and exhaustion or increase mobility, such as walking frames or wheelchairs, are also considered orthopedic devices. Many of these devices have at least one artificial joint to enable a movement of various parts of the orthopedic device, presently the first part and the second part, relative to each other.

A joint according to the preamble in the form of an ankle joint is known, for example, from EP 1 933 775 B1. An active actuator is, for example, a motor, particularly an electric motor, a hydraulic pump or an otherwise actively operable element. With ankle joints according to the preamble, the actuator is used to change an angle between the lower leg part, which constitutes the first part, and the foot part, which constitutes the second part, i.e. to pivot the foot part about the pivot axis relative to the lower leg part. This is practical, for example, when the wearer of the ankle joint is wearing shoes of different heel heights. The higher the heel, the greater the plantar flexion of the foot part must be to produce as natural a gait as possible. During plantar flexion, the forefoot area is lowered and the previously mentioned angle between the foot part and the lower leg part increases.

A joint according to the preamble has a self-locking transmission. A transmission is self-locking when it can be driven via the input shaft, but not via the output shaft. The active actuator is configured to drive the input shaft of the transmission and thus cause a movement of the second part relative to the first part. External forces acting on the second part act on the output shaft of the transmission and are therefore unable to cause a movement due to the self-locking effect of the transmission. This is advantageous in an artificial ankle joint, for example, when the artificial ankle joint enables an adjustability of the heel height, but otherwise remains rigid when walking with a prosthetic foot arranged on an artificial ankle joint and does not allow any further movement of the foot part relative to the lower leg part. Due to the self-locking of the transmission, this is achieved without the actuator having to be driven or actively locked or held. This provides increased fail-safety that ensures sufficient stability of the artificial ankle joint, even in the event that the actuator does not function or does not function safely, for example, if a power supply is faulty or fails. The wearer of the ankle joint and the prosthesis connected thereto is therefore not at risk of losing their stability, on which they are reliant, in the event of such a malfunction. With other joints of orthopedic devices, such as artificial knee joints, this embodiment is also advantageous as, in the event of a failure of the actuator, for instance w % ben the power supply fails, the joint is blocked and buckling thus prevented. In addition, the use of a self-locking transmission is largely more energy efficient, as the joint remains in its position without the support of the motor and the motor need only be active in the event of a movement.

Other ankle joints, likewise according to the preamble, are configured to trigger movements of the foot part relative to the lower leg part while walking or during other movement sequences carried out by the wearer of the artificial ankle joint in order to produce as natural a gait as possible. For example, it is practical to carry out a dorsal flexion during the swing phase of a step, i.e. to raise to forefoot of the prosthetic foot. The angle between the foot part and the lower leg part of the artificial ankle joint is reduced as a result. Depending on the pattern of movement and type of continued movement of the wearer of the prosthesis, other movements may also be advantageous. With such an embodiment of the ankle joint, the actuator is consequently used considerably more frequently, so that a larger energy store, in particular a rechargeable battery, must be provided. The artificial ankle joint is heavier as a result and requires a relatively large installation space.

The prior art also includes ankle joints in which the transmission used is not self-locking. In this case, the actuator is required, on the one hand, to cause a movement of the foot part relative to the lower leg part against externally acting forces and, on the other hand, to prevent the foot part from being able to move relative to the lower leg part when such a movement is not required. Such a configuration is especially disadvantageous in the event of a functional error or failure of the actuator. Whereas with a self-locking transmission an uncontrolled movement of the foot part relative to the lower leg part is prevented by the transmission if the actuator fails, such a movement cannot be prevented when the transmission does not perform this function. In addition, mechanical motion stops often have to be used to determine the range of motion of such a joint. The disadvantage is that the range of motion determined in this way cannot be adjusted. However, the use of a non-self-locking transmission is beneficial when situations arise in which the foot, i.e. In particular the foot part of the artificial ankle joint, is to follow externally acting forces as freely as possible and is to perform corresponding movements relative to the lower leg part.

The invention aims to further develop a method for controlling the joint of an orthopedic device in such a way that the advantages of a self-locking transmission can be combined with those of a non-self-locking transmission.

The invention solves the addressed task by way of a method according to the preamble of claim 1, characterized in that, with the method, the electric control unit controls the actuator in such a way that the second part moves corresponding to forces acting on it from the outside. The electric control unit is thus configured to control the actuator accordingly. This does not mean that the electric control unit always controls the actuator in such a way that the second part moves according to forces acting on it from the outside. However, it is possible that the electric control unit always controls the actuator in this manner. For a method according to the invention, however, it is sufficient if the electric control unit controls the actuator in this way for some of the time.

Forces acting on the second part from outside may, in the case of an artificial ankle joint, be ground reaction forces, for example, which occur when the user of the artificial ankle joint is in contact with the ground via a prosthetic foot arranged on the foot part, which constitutes the second part. If in this case a movement of the foot part relative to the lower leg part, which constitutes the first part, that follows the acting forces is desired, the control unit is used to control the actuator in such a way that the foot part carries out the corresponding movements. Here, it is not necessary, but is advantageous, if the actuator is controlled in such a way that the foot part moves relative to the lower leg part as if it were connected to the lower leg joint by a free-moving joint. It may also be advantageous if the electric control unit controls the actuator in such a way that the foot does indeed move according to the externally acting forces, but does so in a damped manner, in particular against a resistance caused by the actuator and/or the transmission.

The electric control unit preferably comprises an electronic data processing device, in particular a microprocessor, that is configured to control the active actuator and to generate control signals that are transmitted to the actuator. The control signals are preferably generated on the basis of sensor data that are transmitted to the electronic data processing device. Preferably, the electronic data processing device has access to an electronic memory in which, for example, target values or empirical values are stored that are required for generating the control signals and which must be accessible to the electronic data processing device.

The transmission preferably features a first transmission element and a second transmission element that lie against each other and the static friction of which causes the self-locking effect. For example, the first transmission element and the second transmission element can be two threads that engage with one another, such as an inner thread of a first component and an outer thread of a second component. The use of a spindle or a coil as one of the two transmission elements is also possible with, for example, a gearwheel as a second transmission element engaging therein. The first transmission element is driven by the actuator. In this case, the movement is transmitted to the second transmission element and thus also to the output shaft of the transmission, thereby causing a movement by which the second part is pivoted about the pivot axis relative to the first part. However, if an externally acting force is applied to the second part, this force is transmitted to the output shaft of the transmission which, due to the self-locking effect of the transmission, does not lead to a movement.

The first transmission element and the second transmission element lie against each other. The static friction between the two transmission elements depends on different factors, for example the pitch of a thread, the materials used and/or the surface roughness, depending on the design of the elements. However, it also depends on the compressive force with which the two transmission elements are pressed against each other at their contact surfaces. The stronger the force, the higher the static friction and the stronger the self-locking effect. The transmission is preferably designed in such a way that the self-locking effect is only caused by static friction and the sliding friction is small enough to also enable movements due to external forces. In this case, the actuator only has to overcome the static friction once to cancel the self-locking effect. If the two transmission elements are in motion relative to each other, a movement, possibly a damped movement, is possible due to external forces. This applies to this embodiment as long as the transmission remains in motion. This variation renders it possible to minimize the activity required of the motor and thus to save energy. Alternatively, the transmission is preferably designed in such a way that the self-locking effect is ensured both by the static friction and the sliding friction. In this case, the self-locking effect can only be overcome or cancelled with the support of the actuator, even during movement. This variation makes it easier to control the system more precisely and increases safety.

Preferably, the joint is an artificial ankle joint, the first part a lower leg part and the second part a foot part. Alternatively, the joint is an artificial knee joint, the first part an upper leg part and the second part a lower leg part.

If the joint is an ankle joint, it is advantageous to control the actuator depending on a gait phase of a step that is, for example, detected and determined via at least one sensor. In a preferred embodiment, a damped movement is possible in an early stance phase, for example until the foot is fully in contact with the ground. In a second part of the stance phase, on the other hand, the transmission is preferably blocked by the actuator, for example to charge a spring element of a forefoot, such as a spring made of carbon fiber composite, with potential energy. When the load is relieved, this spring is discharged again and the energy is released in the late stance phase. In the swing phase of a step, on the other hand, the foot should preferably be returned to a predetermined position, for example to ensure increased ground clearance and/or to bring the foot into a desirable position for the upcoming initial contact between the heel and the ground.

However, if the joint is an artificial knee joint, it is advantageous to block the joint in the direction of flexion during the stance phase of a step or to allow a damped movement in order to absorb the user's weight. During the swing phase, on the other hand, the knee joint is preferably free to swing, so that the locking effect of the transmission is preferably lifted by the control of the actuator.

Preferably, the position of an ankle joint in which the joint moves from a damped into a blocked state is determined depending on measurement data from the surrounding environment. This relates, for example, to a slope of the ground on which the wearer of the orthopedic device moves or the height of a heel of the shoes worn by the wearer.

Preferably, the actuator is controlled in such a way that the self-locking effect is almost or completely overcome by the actuator when the wearer wishes to move it by themselves into a position that is comfortable, pleasant and ergonomic. This is the case when sitting, for example, when a hip joint, which is likewise a joint that can be controlled by a method according to the invention, should exhibit an angle of approximately 90°.

Preferably, the joint has at least one sensor by which measured values can be detected that allow a statement to be made about the compressive force and/or the static friction between the two transmission elements. For example, there may be a pressure sensor in a contact surface of the first transmission element and/or the second transmission element that is subjected to a pressure which corresponds to the compressive force between the two transmission elements. Alternatively or additionally, a sensor can be used, for example, which determines whether the two contact surfaces of the transmission elements are in contact with each other.

In a preferred embodiment, at least one load measurand is detected by means of at least one sensor that allows a statement to be made about the load on the transmission and/or the static friction between the first transmission element and the second transmission element, wherein the electric control unit controls the actuator depending on the load measurand detected. Preferably, the at least one sensor for detecting the load measurand is part of the joint, especially preferably part of the transmission. However, this is not essential. Sensors outside of the joint can also be used to determine the load measurand and make the corresponding measured values available to the electric control unit.

Advantageously, at least one force measurand is detected by means of at least one sensor that allows a statement to be made on the external forces acting on the second part, wherein the electric control unit controls the actuator depending on the force measurand detected. The at least one sensor for determining the force measurand is preferably part of the joint, preferably part of the second part. However, this is not essential. Sensors outside of the joint can also be used to determine the force measurand and make the corresponding measured values available to the electric control unit.

In a preferred embodiment, a resulting movement and/or a resulting position of the second part relative to the first part is calculated from the force measurand detected. The electric control unit preferably controls the actuator in such a way that the resulting movement is carried out and/or the resulting position is reached. The force measurand detected is preferably made available to the electric control unit, which calculates a resulting movement and/or a resulting position of the second part relative to the first part. To this end, it preferably utilizes calculation specifications, algorithms and software elements stored in an electronic memory to which it has access. In addition or as an alternative, the electric control unit utilizes parameter values which, for example, correspond to a damping, a friction or another variable opposing a movement and which are to be used as a basis for the calculation. For example, for the calculation of a resulting movement, it is important to know whether a movement is damped and, if so, how strongly as well as which forces opposing a movement have to be overcome by forces acting externally, which are characterized by the force measurand. Here, control is preferably conducted in such a way that the actuator overcomes the self-locking effect and the acting external forces provide for the actual movement. Alternatively, however, the actuator can also be controlled in such a way that it both overcomes the self-locking effect and causes the movement that was calculated on the basis of the measured external forces.

Advantageously, the electric control unit can be brought into a first mode and a second mode. In the first mode, it controls the actuator such that the foot part is moved accordingly by forces acting on it from outside. In the first mode, the electric control unit preferably ensures that the self-locking effect of the transmission is cancelled. In addition, damping can optionally be applied to counteract free movement of the joint. In the first mode, the actuator is therefore not controlled towards a target value. A movement of the joint in this mode of the electric control unit is not caused by the electric control unit moving the actuator. Instead, the electric control unit enables the actuator to react to forces acting on it externally and to be moved by these forces.

In the second mode, on the other hand, it controls the actuator independently of such forces. Preferably, the control unit is brought into the first mode when predetermined movements, movement patterns and/or states of movement have been detected and/or when an actuation element has been actuated. In this second mode, the actuator is controlled by the electric control unit in such a way that it moves independently of external forces. Of course, this only applies if the externally acting forces are not greater than the force that can be applied by the actuator.

In a preferred embodiment of the method, the electric control unit is brought into the second mode when a predetermined criterion is met. The criterion is preferably met when an angle between the second part and the first part leaves a predetermined angle range; when the predetermined movements, movement patterns and/or states of movement are not or are no longer detected; when an actuation element has been actuated and/or after the electric control unit has been in the first mode for a predetermined period of time. By switching the operating mode into the second mode when a predetermined limit angle between the second part and the first part is reached, the range of motion of the joint can be restricted. In this embodiment, this does not require any mechanical stops to be moved or the joint to be disassembled and re-assembled in a different way. Instead, it is enough to adjust the limit angle stored as a parameter in the electronic memory.

Particularly preferably, the criterion can be adjusted or changed. This can be done, for example, by the wearer or a third party, such as an orthopedic technician who performs adjustments to the joint. At least one actuation element or adjustment element can be provided on the joint for this purpose. This is advantageous, for example, when the criterion is an angle between the two joint parts. This limit angle can thus be easily adjusted. Alternatively or additionally, the criterion can be adjusted by means of a software. In this case, the criterion is stored in a software that runs in the electric control unit of the joint, particularly an electronic data processing device, and is executed by said control unit. Via a communication connection between the electric control unit of the joint and a further electronic data processing device, the software and the criterion stored therein can be accessed and the criterion changed. In the process, at least one parameter can be adjusted, changed or selected, or the criteria itself changed or exchanged.

The invention also solves the addressed task by way of a joint, especially an ankle joint, that is suited to carry out a method described here.

It preferably has at least one sensor for detecting a load measurand, which comprises at least one expansion measuring strip, a spring force measure, a deformation sensor, a torque sensor, a pressure sensor and/or an axial load sensor.

Preferably, the ankle joint features at least one sensor for detecting the force measurand, which comprises at least one force sensor, a position sensor, an inertial sensor and (or a gyroscope.

In the following, a number of embodiment examples of the invention will be explained in more detail with the aid of the accompanying figures. They show

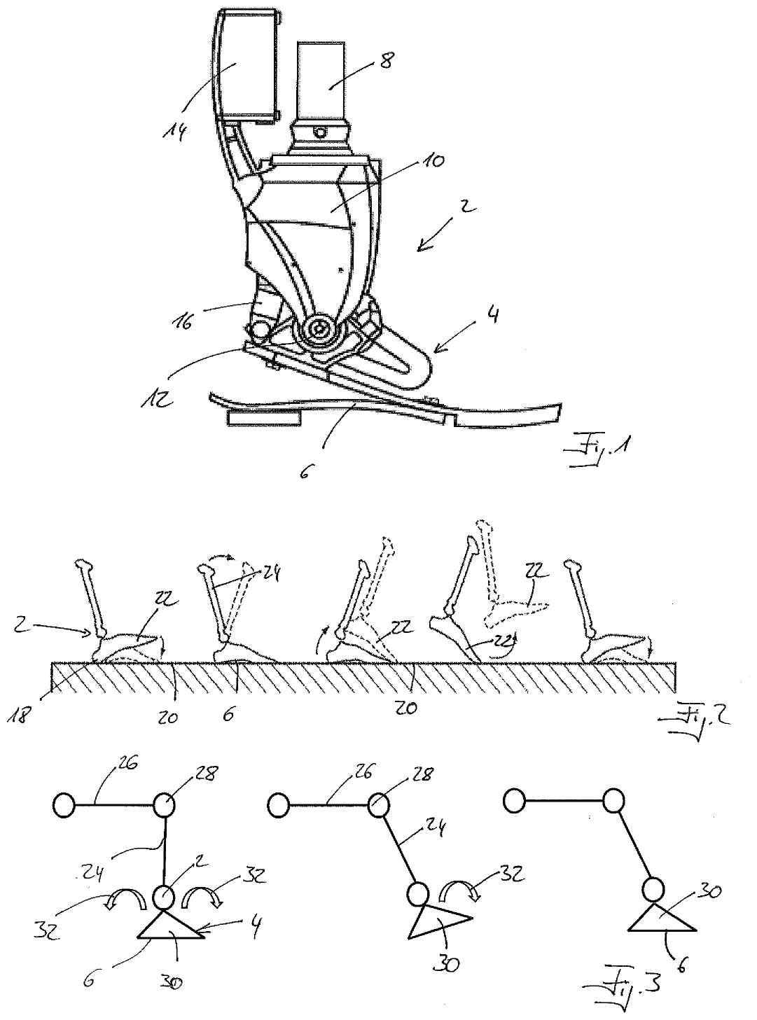

FIG. 1—a schematic representation of a prosthetic foot with an ankle joint according to an embodiment example of the present invention.

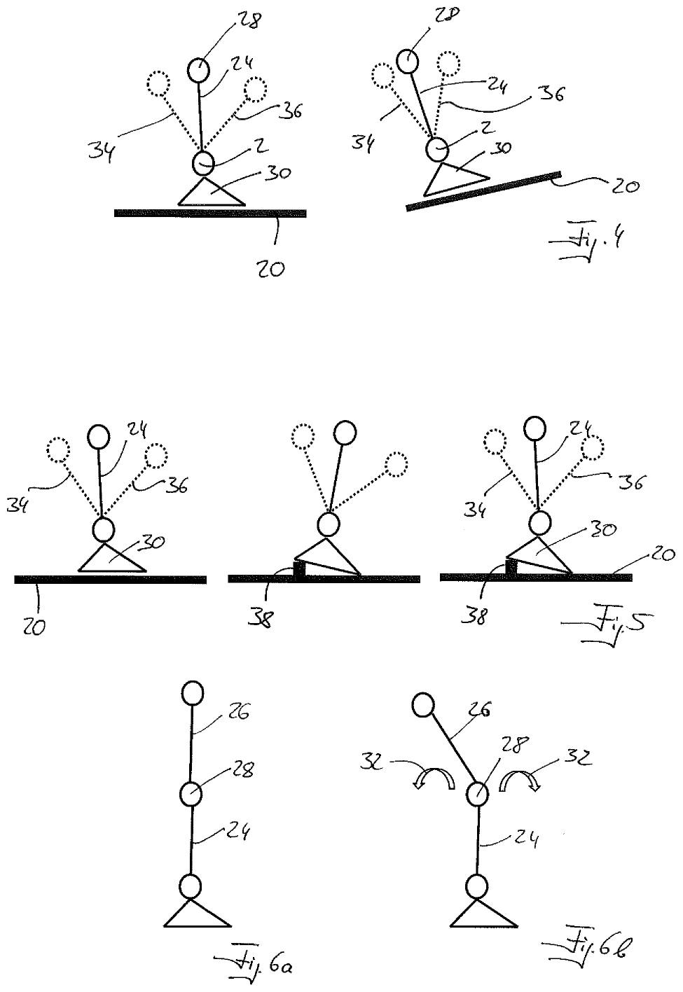

FIG. 2—schematic phases of a gait cycle,

FIG. 3—schematic movements and positions of a leg w % bile sitting,

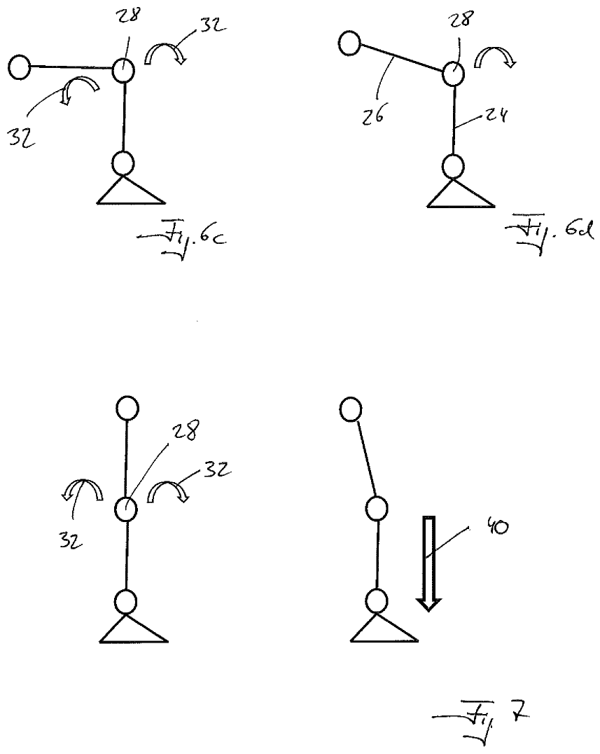

FIG. 4—schematic representations of the range of motion whilst walking in a sloped plane,

FIG. 5—schematic representations of the scope of movement of an ankle at different heel heights,

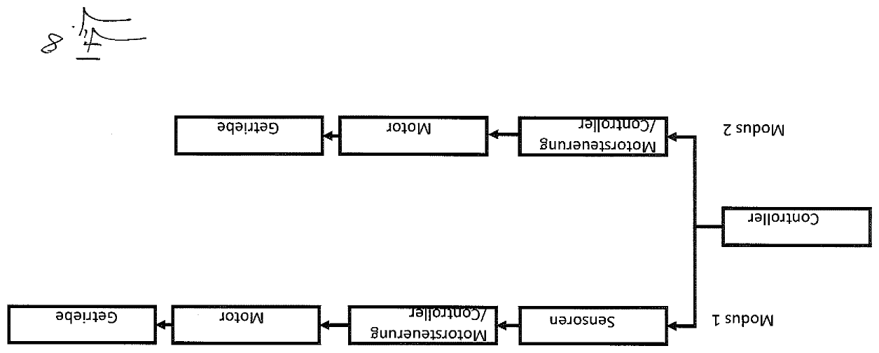

FIGS. 6a-6d—various schematic positions of a leg while sitting down and standing up,

FIG. 7—schematic representations of the control of a knee joint at various loads,

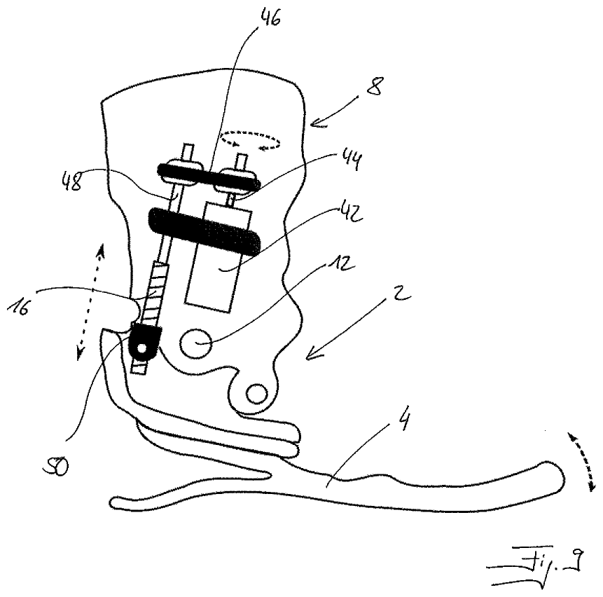

FIG. 8—a schematic representation of the control unit in different modes, and

FIG. 9—a schematic sectional view through a joint according to an embodiment example of the present invention.

FIG. 1 schematically depicts a prosthetic foot with an active ankle joint 2, which is designed according to an embodiment example of the present invention. It connects a second part 4, designed as a prosthetic foot with a foot base 6, to a first part 8, which is designed as an adaptor element on which a lower leg element can be arranged. A housing 10 contains a self-locking transmission and an actuator as well as an electric control unit, which is configured to conduct a method described here. The second part 4 is arranged on the first part 8 such that it can be pivoted about a pivot axis 12. The actuator, designed as a motor, is supplied with energy via a battery 14. In the embodiment example shown, the motor is able and configured to displace a spindle 16 upwards and downwards, and thus to change a pivot angle between the second part 4 and the first part 8.

FIG. 2 schematically shows four phases of a gait cycle. The first phase in the far left-hand representation in FIG. 2 corresponds to the first phase of the next step in the far right-hand representation in FIG. 2. This first phase is the so-called heel strike. The ankle joint 2, depicted only schematically, is in principle designed in the same way as the joint shown in FIG. 1. A heel 18 comes into contact with a ground 20. In this phase, the joint is operated in the first mode, so that the electric control unit controls the actuator in such a way that the second part 4 moves in accordance with the externally acting forces. Said forces cause a forefoot 22 to lower until the foot base 6 is fully on the ground 20. The respective phase of the gait cycle is determined via sensors, which can be arranged at various positions of the prosthetic foot and/or the ankle joint 2. The electric control unit is brought into the first or second mode on the basis of the sensor data.

In the second representation from FIG. 2, the rollover phase is shown, in which the foot base 6 lies fully on the ground 20 and a lower leg 24 moves forward. In all the phases depicted in FIG. 2, the movement is from the position indicated by a solid line into the position indicated by a dashed line. In this case too, the electric control unit is operated in the first mode, so that the actuator cancels the self-locking effect of the transmission and the second part 4 moves relative to the first part 8 as if it were being moved by the externally acting forces.

In the third representation in the middle of FIG. 2, the phase of pushing off from the ground 20 is shown. Sensors detect that a predetermined dorsal stop is reached, i.e. an angle between the first part 8 and the second part 4 assumes a predetermined value. In the embodiment example shown, the electric control unit is then brought from the first mode into the second mode, so that the self-locking effect of the transmission is no longer cancelled. The joint no longer moves according to the forces acting externally on the joint, but blocks, so that the foot can push off from the ground 20.

In the penultimate representation in FIG. 2, the swing phase is shown in which the foot loses contact to the ground. In the process, the forefoot 22 is raised, wherein the position reached during this movement is pre-set. The movement is caused by the actuator, i.e. the motor in the present case. In an especially preferred embodiment, an active plantar flexion of the foot, i.e. a lowering of the forefoot 22 and therefore an active push-off, is carried out when the foot is pushing off from the ground 20. This increases an angle between the second part 4 and the first part 8 at which the actuator moves the second part 4 relative to the first part 8. If this is the case, it is advantageous to raise the forefoot again in the swing phase by way of a dorsal flexion and to reach the desired position for the next heel strike. Alternatively, it is also possible to not carry out a plantar flexion when the foot is pushing off. In this case, it is not necessary, but indeed advantageous, to carry out a dorsal flexion during the swing phase.

In the embodiment example shown, sensors, such as pressure sensors, are arranged on the foot base 6 or load sensors at different points on the ankle joint 2 by which, as is generally known from the prior art, different phases of a gait cycle can be detected. Depending on whether a free movement of the second part 4 relative to the first part 8 is desired, the electric control unit is brought into the first mode or the second mode.

FIG. 3 schematically shows the representation of a leg prosthesis with an upper leg 26, a knee 28, a lower leg 24, an ankle joint 2 and a foot 30. The ankle joint 2 is configured to be controlled according to a method in accordance with an embodiment example of the present invention. The left-hand representation in FIG. 3 shows the situation in which the wearer of the prosthesis is sitting. The knee 28 is almost at a right angle and the foot base 6 of the foot lies fully on the ground. In this situation, it is beneficial to operate the electric control unit in the first mode, so that the second part 4, i.e. the foot 30 in the present example, can move according to the externally acting forces. This is schematically depicted by the two arrows 32.

The middle representation in FIG. 3 shows that the wearer of the prosthesis is pivoting the lower leg 24 relative to the upper leg 26, so that the knee 28 exhibits a greater angle. The foot 30 is slightly raised, but has not changed its angle relative to the lower leg 24. In the right-hand representation from FIG. 3, the foot 30 touches down and moves along the arrow 32 from the middle representation in FIG. 3, so that the foot base 6 once again lies fully on the ground. This is possible because the electric control unit is operated in the first mode and the actuator controlled in such a way that the second part 4 moves corresponding to the externally acting forces relative to the first part 8. As a result, the user can at all times adjust to a position that is comfortable for them. This would not be possible by controlling the ankle position, as is known from the prior art. Here, the ankle position would be adjusted solely via the motor, but in this case the information on the desired position would be missing.

FIG. 4 schematically shows the influence of the slope of a ground 20 on which the wearer of the prosthesis is walking. Again, a leg prosthesis with the lower leg 24, the knee 28, the ankle joint 2 and the foot 30 is schematically depicted, the ankle joint 2 again being configured to be controlled according to a method for controlling the joint in accordance with an embodiment example of the present invention. The foot 30 forms the first part 8 and the lower leg 24 forms the second part 4. In each case, dashed lines depict a plantar stop 34 and a dorsal stop 36, which indicate the maximum range of motion of the ankle joint. In the left-hand representation in FIG. 4, the wearer of the prosthesis is standing on an even and horizontal surface; in the right-hand representation in FIG. 4 the ground 20 is sloped. This changes the range of motion required between plantar stop 34 and dorsal stop 36. During the phases 1 to 3 of the gait cycle, which are shown in FIG. 2, the ankle joint 2 moves in such a way that the foot 30 is moved relative to the lower leg 24 within this range of motion. In this range, the electric control unit is operated in the first mode, so that a self-locking effect of the transmission is cancelled. As soon as one of the stops 34, 36 is reached, which is detected via sensors, for example, and passed on to the electric control unit, the electric control unit is brought from the first mode into the second mode, so that the self-locking effect of the transmission is not cancelled. If, for example, the slope of the ground 20 is now determined via further sensors, the actual value of the angle can be adjusted and changed for the plantar stop 34 and/or the dorsal stop 36.

FIG. 5 shows the influence of a heel height of a schematically depicted heel 38 of a shoe. The left-hand representation in FIG. 5 corresponds to the left-hand representation in FIG. 4. The foot 30 lies fully on the ground 20 and the dorsal stop 36 and the plantar stop 34 limit the range of motion that the lower leg 24, i.e. the first part 8, has relative to the foot 30, i.e. the second part 4, when the electric control unit is operated in the first mode. If the wearer of this prosthesis now puts on a shoe with a heel 38, there is no initial change to the range of motion and the actual values of the various stops 34, 36. This is shown in the middle representation of FIG. 5. However, the change in heel height causes a change, for example, to the angle between the foot 30 and the lower leg 24 at which the heel of the foot 30 comes into contact with the ground 20 during the heel strike. If the heel height is detected via a sensor, the stops 34, 36, which are not mechanical stops, but rather simply electronic or virtual stops, can be adjusted. This is shown in the right-hand representation of FIG. 5.

FIGS. 6a to 6d depict various situations during sitting down and standing up with a leg prosthesis. It has the upper leg 26 and the lower leg 24, between which the knee 28 is located. In FIG. 6, the knee 28 is suitable and configured to be controlled according to the present invention. FIG. 6a depicts an extended leg such as occurs, for example, during standing and walking, especially for patients with low degrees of mobility. In this case, the knee joint 28 is preferably blocked and consequently the self-locking effect of the transmission is not cancelled. The electric control unit is operated in the second mode.

In FIG. 6b, sensors have detected, for example, that the wearer of the prosthesis wants to sit down. To this end, it is advantageous for the self-locking effect of the transmission, which is located in the knee joint 28, to be cancelled, so that the knee joint 28 can move according to the externally acting forces. This is possible in both directions, which is schematically depicted by the arrows 32.

FIG. 6c shows the situation when sitting. The electric control unit remains in the first mode, like in FIG. 6b, and the knee joint 28 can move freely along the two arrows 32. FIG. 6d, on the other hand, depicts the process of standing up. This can also be detected via sensors, for example. When standing up, it is advantageous if the knee joint 28 supports the wearer of the prosthesis while they are standing up. The self-locking effect is consequently active and the actuator is controlled by the electric control unit in such a way that a desired end position is reached. The knee joint is controlled as the active knee joint it is. In addition, the self-locking effect prevents a renewed, unwanted flexion if the active control of the joint happens to fail, so that the knee joint depicted is secure in all situations.

FIG. 7 shows a way of identifying whether the electric control unit is being operated in the first mode, as depicted in the left-hand representation of FIG. 7, or in the second mode. For example, if only a small load is detected on the prosthetic leg, the self-locking effect is cancelled and the electric control unit operated in the first mode. The knee joint 28 can be moved along the arrows 32 in both directions according to the externally acting forces. The situation is different when a large load is acting on the prosthetic leg, as is shown in the right-hand representation in FIG. 7 by the arrow 40. Under this high load, cancelling the self-locking effect of the transmission would be a safety hazard for the wearer of the prosthesis, so that the electric control unit of the joint is operated in the second mode.

FIG. 8 schematically shows how the control unit works in the two different modes. A controller first determines, in the electric control unit or a separate electric control unit, whether the electric control unit is operated in the first mode, i.e., the upper string in FIG. 8, or in the second mode, i.e., the lower string of FIG. 8, based on sensor data recorded by sensors, not depicted. In the upper string, the externally acting forces are detected via sensors and evaluated in the electric control unit, i.e. the motor control unit or a controller. The motor, i.e. the actuator, is then controlled in such a way that it cancels the self-locking effect of the transmission and allows a movement according to the externally acting forces.

In the lower string of FIG. 8, in which the electric control unit is operated in the second mode, it is not necessary to detect the externally acting forces in order to control the actuator. Here, the self-locking effect of the transmission is active and the actuator or motor is controlled in such a way that a desired position is reached or maintained.

FIG. 9 shows a schematic sectional view through a prosthetic foot with an ankle joint 2, a first part 8 and a second part 4. The second part 4 is arranged about a pivot axis 12 on the first part 8. An active actuator 42 in the form of a motor is arranged on the first part 8, said actuator being configured to rotate a first shaft 44. In the embodiment example shown, the rotation of the first shaft 44 is transmitted via a timing belt 46 to a second shaft 48, which likewise is set in rotation. The spindle 16, which comprises an outer thread, is located on said second shaft. A screw sleeve 50 is located on the second part 4, the former comprising an inner thread designed to correspond to the outer thread of the spindle 16. Together, the spindle 16 and the screw sleeve 50 form a self-locking transmission.

### REFERENCE LIST

2 ankle joint
4 second part
6 foot base
8 first part
10 housing
12 pivot axis
14 battery
16 spindle
18 heel
20 ground
22 forefoot
24 lower leg
26 upper leg
28 knee
30 foot
32 arrow
34 plantar stop
36 dorsal stop
38 heel
40 arrow
42 active actuator
44 first shaft
46 timing belt
48 second shaft
50 screw sleeve

1. A method for controlling a joint (2, 28) of an orthopedic device that comprises a first part (8), a second part (4), which is arranged on the first part (8) such that it can be pivoted about a pivot axis (12), an active actuator (42), a self-locking transmission (16, 50) and an electric control unit for controlling the actuator (42), characterized in that during the method the electric control unit controls the actuator (42) in such a way that the second part (4) moves according to forces acting on it externally.

2. The method according to claim 1, characterized in that the joint (2) is an artificial ankle joint (2), the first part (8) is a lower leg part and the second part (4) a foot part.

3. The method according to claim 1, characterized in that the joint is an artificial knee joint (28), the first part (8) is an upper leg part and the second part (4) a lower leg part.

4. The method according to claim 1, characterized in that at least one load measurand is detected by means of at least one sensor that allows a statement to be made about the load on the transmission (16, 50) and/or the static friction between the first transmission element (16) and the second transmission element (50), wherein the electric control unit controls the actuator (42) depending on the load measurand detected.

5. The method according to claim 1, characterized in that at least one force measurand is detected by means of at least one sensor that allows a statement to be made about the external forces acting on the second part (4), wherein the electric control unit controls the actuator (42) depending on the force measurand detected.

6. The method according to claim 5, characterized in that a resulting movement and/or a resulting position of the second part (4) relative to the first part (8) is calculated from the force measurand detected and the electric control unit controls the actuator (42) in such a way that the resulting movement is carried out and/or the resulting position is reached.

7. The method according to claim 1, characterized in that the electric control unit can be brought into a first mode and into a second mode, wherein in the first mode it controls the actuator (42) in such a way that the second part (4) is moved according to forces that act on it externally and do not do so in the second mode.

8. The method according to claim 7, characterized in that the electric control unit is brought into the first mode when predetermined movements, movement patterns and/or states of movement have been detected and/or when an actuation element has been actuated.

9. The method according to claim 7, characterized in that the electric control unit is brought into the second mode when a predetermined criterion is met.

10. The method according to claim 9, characterized in that the criterion is met when an angle between the second part (4) and the first part (8) leaves a predetermined angle range; when the predetermined movements, movement patterns and/or states of movement are not or are no longer detected; when an actuation element has been actuated and/or after the electric control unit has been in the first mode for a predetermined period of time.

11. The method according to claim 9, characterized in that the predetermined criterion can be adjusted or changed.

12. A joint (2, 28) for an orthopedic device for carrying out a method according to claim 1.

13. The joint according to claim 12, characterized in that it has at least one sensor for detecting a load measurand, which comprises at least one expansion measuring strip, a spring force measure, a deformation sensor, a torque sensor, a pressure sensor and/or an axial load sensor.

14. The joint according to claim 13, characterized in that it has at least one sensor for detecting the force measurand, which comprises at least one force sensor, a position sensor, an inertial sensor and/or a gyroscope.
